(19) United States
(12) Patent Application Publication
(10) Pub. No.: US 2013/0023925 A1
(43) Pub. Date: Jan. 24, 2013

(54) Articulating Surgical Apparatus

(71) Applicant: Peter M. Mueller, Frederick, CO (US)

(72) Inventor: Peter M. Mueller, Frederick, CO (US)

(73) Assignee: Tyco Healthcare Group LP, Boulder, CO (US)

(21) Appl. No.: 13/186,695

(22) Filed: Jul. 20, 2011

(51) Int. Cl. A61B 17/28

(52) U.S. Cl. 606/205

(57) ABSTRACT

An endoscopic instrument includes a housing having shaft extending therefrom that defines a longitudinal axis therethrough. The shaft includes an articulating portion disposed thereon. An end effector assembly operatively connected to a distal end of the shaft configured to treat tissue includes a pair of first and second jaw members. A locking tube is coaxially disposed on the shaft is movable along the longitudinal axis. The locking tube is movable along an outer surface of the shaft from a retracted position such that the shaft may be articulated transversely across the longitudinal axis, to an extended position such that the shaft is locked in a fixed position along the longitudinal axis.

### BACKGROUND

1. Technical Field

The present disclosure relates to an articulating surgical apparatus. More particularly, the present disclosure relates to an articulating surgical apparatus including a locking tube configured to lock the surgical apparatus in a non-articulated configuration.

2. Description of Related Art

Surgical instruments that are configured to articulate or bend are well known in the medical arts. Surgical instruments of this nature are utilized in many surgical procedures. For example, laparoscopic, endoscopic, or other minimally invasive surgical procedures are just a few of the many surgical procedures where articulating surgical instruments may find use. When utilized in such procedures, the surgical instruments may include a housing, a handle assembly, an articulating shaft, a device for articulating the shaft, and an end effector including a pair of jaw members.

As can be appreciated, the relatively small operable working space that is created within a cavity of a patient during a surgical procedure often makes it difficult for the surgeon to position the jaw members adjacent or close to target tissue. The articulating shaft allows a surgeon to position the jaw members adjacent target tissue.

Various articulating devices or mechanisms may be utilized to articulate the shaft. For example, some surgical instruments utilize one or more articulating cables or tendons that couple to one or more articulation links on the shaft. Typically, the cables or tendons provide a mechanical interface from the one or more articulation links to an actuation device, e.g., rotatable dials, disposed on the housing and/or handle assembly of the surgical instrument such that actuation of the actuation device moves or articulates the shaft about the articulation links. In particular, when the cables or tendons are “pulled” or otherwise manipulated via one or more mechanisms in the handle assembly or the housing to articulate the shaft about the articulating links.

Under certain surgical scenarios, it may prove advantageous to maintain the shaft in a relatively fixed or stationary position, such as, for example, when positioning tissue between the jaw members or when the shaft is inserted through a trocar or cannula. Locking the cables or tendons so that the shaft is prevented from articulating typically requires eliminating, what is commonly referred to in the art as, cable or tendon “stretch” from the cables or tendons. Cable or tendon “stretch” is the ability of the cable or tendon to stretch under a predetermined load. To remove this cable or tendon stretch, the cables or tendons are typically highly loaded in tension. Removing this cable or tendon stretch limits and/or eliminates “post lock” articulation. However, due to the length of the surgical instrument and, thus, the corresponding length of the cables or tendons between the articulating links and the actuation device and/or locking device, a fairly large “spring rate” exists with a corresponding “stiffness” penalty being observed. That is, overtime, subjecting the cables or tendons to high load tension reduces the stiffness of the cables or tendons and, thus, the overall stiffness of the shaft. As can be appreciated, reducing the “stiffness” of the shaft may result in the shaft not functioning in a manner as intended.

### SUMMARY

The present disclosure provides an endoscopic instrument. The endoscopic instrument includes a housing having shaft extending therefrom that defines a longitudinal axis therethrough. The shaft includes an articulating portion disposed thereon. An end effector assembly operatively connected to a distal end of the shaft includes a pair of first and second jaw members. A locking tube coaxially disposed on the shaft is movable along the longitudinal axis. The locking tube is movable along an outer surface of the shaft from a retracted position such that the shaft may be articulated transversely across the longitudinal axis, to an extended position such that the shaft is locked in a fixed position along the longitudinal axis.

In certain embodiment, one or both of the first and second jaw members is movable relative to other jaw member from an open position, wherein the first and second jaw members are disposed in spaced relation relative to one another, to a clamping position, wherein the first and second jaw members cooperate to grasp tissue therebetween.

The present disclosure provides an endoscopic instrument. The endoscopic instrument includes a housing having shaft extending therefrom that defines a longitudinal axis therethrough. The shaft includes a plurality of articulating links in operable communication with a pair of articulation dials of the endoscopic instrument via a plurality of tendons. An outer diameter of the plurality of articulating links is cocylindrical with shaft. An end effector assembly operatively connected to a distal end of the shaft includes a pair of first and second jaw members. A locking tube coaxially supported on the shaft is selectively movable therealong upon actuation of an actuation device disposed on the housing. The locking tube is movable along the outer surface of the shaft from a retracted position for articulating the shaft transversely across the longitudinal axis, to an extended position for locking the shaft in a fixed position along the longitudinal axis.

In certain embodiment, one or both of the first and second jaw members is movable relative to other jaw member from an open position, wherein the first and second jaw members are disposed in spaced relation relative to one another, to a clamping position, wherein the first and second jaw members cooperate to grasp tissue therebetween.

### BRIEF DESCRIPTION OF THE DRAWING

Various embodiments of the present disclosure are described hereinbelow with references to the drawings, wherein:

FIG. 1 is a side, perspective view of an endoscopic instrument showing a locking device in a retracted position according to an embodiment of the present disclosure;

FIG. 2 is a side, perspective view of the endoscopic bipolar forceps depicted in FIG. 1 showing the locking device in an extended position;

FIG. 3A is a cross-sectional view taken along line segment “3A-3A” depicted in FIG. 1;

FIG. 3B is an enlarged view of the area of detail depicted in FIG. 1;

FIG. 4 is a perspective view of an articulation mechanism according to another embodiment of the present disclosure; and

FIG. 5 is a cross-sectional view taken along line segment “5-5” depicted in FIG. 4.

### DETAILED DESCRIPTION

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

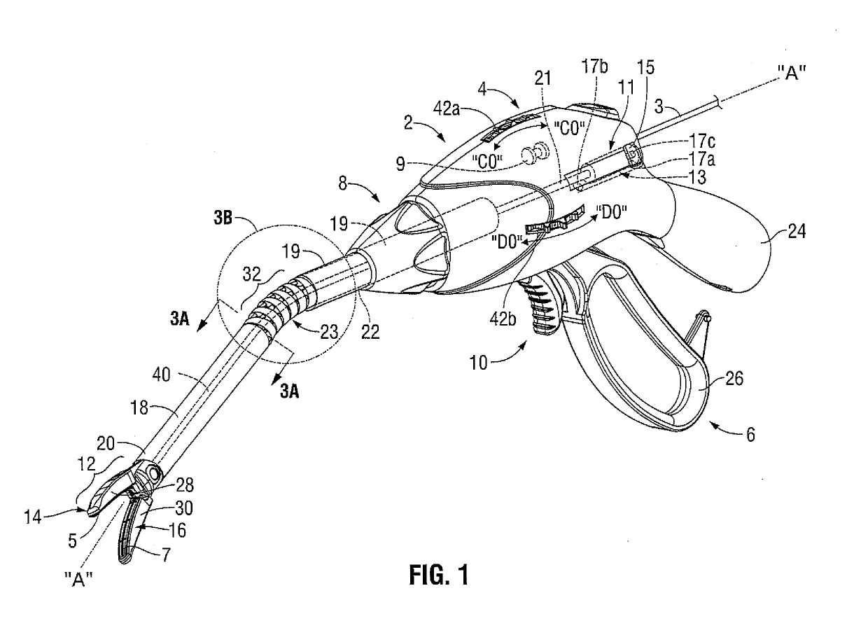

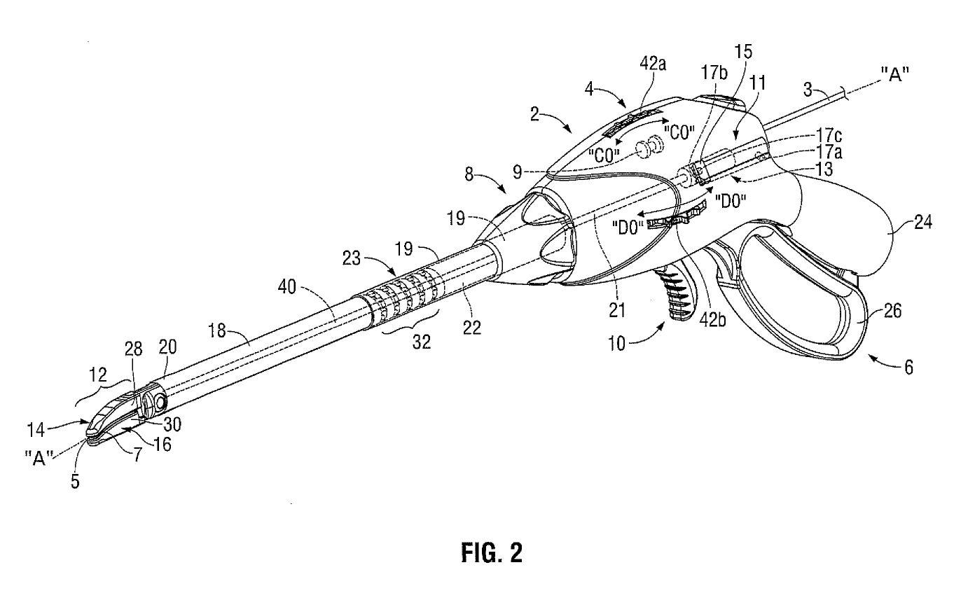

With reference to FIGS. 1 and 2, an illustrative embodiment of an articulating surgical instrument, e.g., an articulating endoscopic instrument, such as, for example, an articulating endoscopic forceps 2 (forceps 2), is shown. As can be appreciated, other types of articulating instruments that are configured to treat tissue may be utilized in accordance with the present disclosure, e.g., snares, blades, loops, endoscopes, stabilizers, retractors, etc. Forceps 2 is configured to operatively and selectively couple to a suitable energy source, such as, for example, an electrosurgical generator (not shown), for performing an electrosurgical procedure. An electrosurgical procedure may include sealing, cutting, cauterizing, coagulating, desiccating, and fulgurating tissue all of which may employ RF and/or microwave energy. The generator may be configured for monopolar and/or bipolar modes of operation. The generator may include or is in operative communication with a system (not shown) that may include one or more processors in operative communication with one or more control modules that are executable on the processor. The control module (not explicitly shown) may be configured to instruct one or more modules to transmit electrosurgical energy, which may be in the form of a wave or signal/pulse, via one or more cables (e.g., an electrosurgical cable 3) to one or both seal plates 5, 7 disposed on respective jaw housings 28 and 30.

Continuing with reference to FIGS. 1 and 2, forceps 2 is shown configured for use with various electrosurgical procedures and generally includes a housing 4, an electrosurgical cable 3 that connects the forceps 2 to a source of electrosurgical energy, a handle assembly 6, a rotating assembly 8, a trigger assembly 10, a drive assembly 9, and an end effector assembly 12 that operatively connects to the drive assembly 9. The drive assembly 9 may be in operative communication with handle assembly 6 for imparting movement of one or both of a pair of jaw members 14, 16 of end effector assembly 12.

For a more detailed description of the rotating assembly 8, trigger assembly 10, and electrosurgical cable 3 (including line-feed configurations and/or connections), reference is made to commonly owned U.S. Pat. Publication No. 2007/0173814 filed on Nov. 9, 2006.

With continued reference again to FIGS. 1 and 2, housing 4 is illustrated. Housing 4 is accessible by a surgeon from outside a body cavity to control the positioning, orientation and operation of the end effector 12 when the end effector 12 is positioned inside a body cavity at a surgical site. To provide this operability, the housing 4 supports various components that are operable to induce or prohibit movement in the end effector 12 through various modes. More particularly housing 4 is configured to house or support handle assembly 6, drive assembly 9, a pair of articulation dials 42a, 42b and an actuation device 11.

Continuing with reference to FIGS. 1 and 2, an elongated slot 13 of suitable configuration is disposed on the housing 4. In the illustrated embodiments, the elongated slot 13 is disposed on a left side of the housing 4 adjacent a stationary handle 24 of the handle assembly 6. Elongated slot 13 is configured to slidably house actuation device 11 (FIGS. 1 and 2) therein such that the actuation device 11 is actuatable via a finger of a user. One or more detents 17a and 17b are operably disposed at proximal and distal ends, respectively, of the elongated slot 13 and are configured to releasably engage a corresponding structure, e.g., an indent 17c, associated with the actuation device 11 (FIG. 1).

Actuation device 11 includes a resiliently-biased slide mechanism 15 (slide mechanism 15) that is operably coupled to the elongated slot 13 on the housing 4 (FIGS. 1 and 2). The slide mechanism 15 is translatable within the elongated slot 13 from a retracted position that corresponds to a locking tube 19 being in the retracted position (FIG. 1), to an extended position that corresponds to the locking tube 19 being in the extended position (FIG. 2). A spring (not explicitly shown) may operably couple to the slide mechanism 15 and may be configured to bias the slide mechanism 15 in a downwardly direction to lock the slide mechanism 15 in one or more positions within the elongated slot 13. In particular, the spring is configured to selectively bias the slide mechanism 15 in a downwardly direction when the slide mechanism 15 is in the retracted and extended positions. An indent 17c (shown in phantom in FIG. 1) is defined on a bottom surface of the slide mechanism and is configured to releasably engage the detents at the proximal and distal ends of the elongated slot 15. More particularly, when the sliding mechanism 15 is moved to the retracted position, the indent 17c moves into releasable engagement with detent 17a (FIG. 1). Likewise, when the sliding mechanism 15 is moved to the extended position, the indent 17c moves into releasable engagement with detent 17b (FIG. 2). The indent/detent configuration facilitates maintaining the sliding mechanism 15 in the retracted and extended positions until a predetermined force is exerted on the sliding mechanism 15 to move the indent 17c out of engagement with either of the detents 17a and 17b.

Actuation mechanism 15 includes or operably couples to an actuation rod 21 (FIGS. 1 and 2) that operably couples to the locking tube 19 via one or more suitable coupling methods including, but not limited to soldering, brazing, spot welding, ultrasonic welding, etc. In the illustrated embodiment, the actuation rod 21 moves the locking tube 19 along an outer surface of a shaft 18 and over an articulation portion 23 of the shaft 18, see FIG. 2 for example.

Referring again to FIGS. 1 and 2, articulation dials 42a, 42b are operable to pivot the distal end 20 of the elongated shaft 18 to various articulated orientations with respect to a longitudinal axis A-A. For example, articulation dial 42a may be rotated in the direction of arrows “C0” to induce pivotal movement in a first plane, e.g., a vertical plane. Similarly, articulation dial 42b may be rotated in the direction of arrows “D0” to induce pivotal movement in a second plane, e.g., a horizontal plane.

Continuing with reference with FIGS. 1 and 2, shaft 18 includes a generally elongated configuration and defines a longitudinally axis “A-A” therethrough. Shaft 18 includes the distal end 20 that is configured to mechanically engage the end effector assembly 12 and a proximal end 22 that mechanically engages the housing 4. In the drawings and in the descriptions that follow, the term “proximal,” as is traditional, will refer to the end of the forceps 2 that is closer to the user, while the term “distal” will refer to the end of the forceps 2 that is farther from the user.

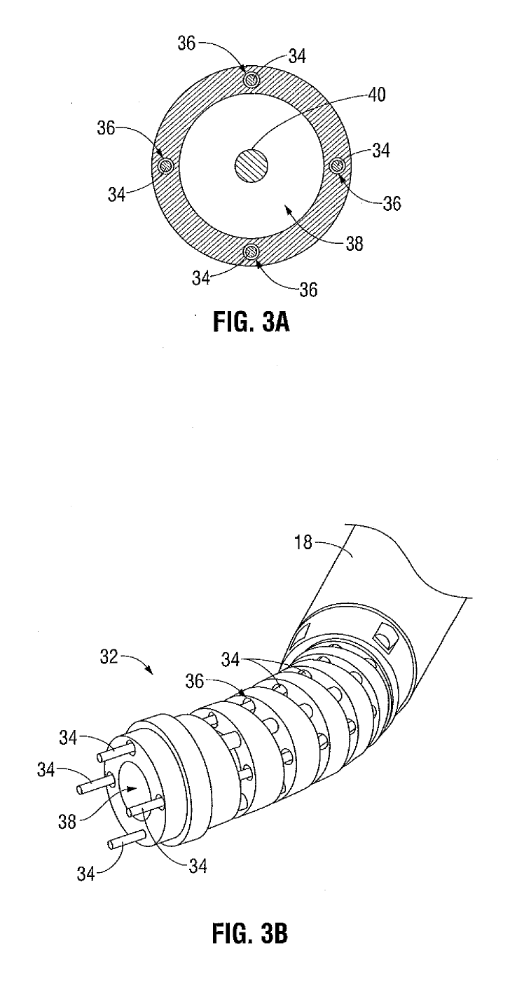

An articulation portion 23 is operably disposed on or coupled to the shaft 18 between the proximal and distal ends 20 and 22, respectively (FIG. 1). In the embodiment illustrated in FIGS. 1-3B, the articulation portion 23 is defined by a plurality of articulating segments or links 32 (links 32), FIGS. 1 and 3B. The links 32 is configured to pivot or articulate the shaft 18 transversely across the longitudinal axis “A-A” in either the horizontal or vertical plane. For illustrative purposes, the shaft 18 is shown articulated across the horizontal plane.

Referring to FIGS. 3A and 3B, the links 32 are operably coupled to the articulation dials 42a and 42b via a plurality of cables or tendons 34 (tendons 34). For illustrative purposes, four (4) tendons are shown (hereinafter collectively referred to as tendons 34). The tendons 34 may be constructed of stainless steel wire or other material suitable for transmitting tensile forces to a distal-most link of links 32. Regardless of the construction materials, the tendons 34 exhibit a spring rate that is amplified over the length of the tendons 34 and thus, the tendons 34 may tend to stretch when external loads are applied to the elongated shaft 18. This tendency to stretch may be associated with an unintended change in orientation of the distal portion 20 of the elongated shaft 18, e.g., without a corresponding movement of the articulation dials 42a, 42b initiated by the surgeon.

The tendons 34 operably couple to the articulating dials 42a and 42b that are configured to actuate the tendons 34, i.e., “pull” the tendons 34, when the articulating dials 42a and 42b are rotated. The tendons 34 operably couple to the links 32 via one or more suitable coupling methods. More particularly, each link of the links 32 includes four (4) corresponding apertures 36 that are radially disposed thereon and centrally aligned along a common axis, see FIG. 3B. The apertures 36 are configured to receive a corresponding tendon of the tendons 34 therein. A distal end of each tendon of the tendons 34 is operably coupled to the distal most link of the links 32 by one or more suitable coupling methods, e.g., one or more of the coupling methods described above.

With reference again to FIG. 3A, the plurality of articulating links 32 collectively define a central passageway 38 configured to receive a drive mechanism, e.g., a drive rod 40 (FIGS. 1-3A), therethrough. As can be appreciated, the configuration of the central passageway 38 provides adequate clearance for the drive rod 40 therethrough.

To facilitate movement of the locking tube 19 along the shaft 18 including the links 32, an outer diameter of the links 32 is cocylindrical with the shaft 18, as best seen in FIGS. 1 and 2. That is, the outer diameter of links 32 is equal to an outer diameter of the shaft 18. In some embodiments, the outer diameter of the links 32 is less than the outer diameter of the shaft 18.

Referring again to FIGS. 1 and 2, the locking tube 19 is illustrated. The locking tube 19 may be made from any suitable material including plastic, metal, etc. In the illustrated embodiment, the locking tube 19 is made from a substantially rigid plastic. The locking tube 19 includes an inner diameter configured to contact an outer circumferential surface of the shaft 18 such that the locking tube 19 forms a tight or “snug” fit around the shaft 18. The locking tube 19 is supported on the shaft 18 and extends partially along a length thereof. A proximal end of the locking tube 19 is positioned within the housing 4 and is configured to operably couple to the actuation rod 21. The locking tube 19 is configured such that in the retracted position, a distal end of the locking tube 19 does not cover any of the links of the links 32 (FIG. 1) and, in the extended position, the distal end of the locking tube 19 covers all of the links of the links 32 (FIG. 2). As can be appreciated, this provides maximum articulation in the retracted position and no or minimal articulation in the extended position.

Continuing with reference to FIGS. 1 and 2, handle assembly 6 includes a fixed handle 24 and a movable handle 26. Fixed handle 24 is integrally associated with housing 4 and movable handle 26 is movable relative to fixed handle 24. Movable handle 26 of handle assembly 6 is ultimately connected to the drive assembly 9, which together mechanically cooperate to impart movement of one or both of the jaw members 14 and 16 to move from an open position (FIG. 1), wherein the jaw members 14 and 16 are disposed in spaced relation relative to one another, to a clamping or closed position, wherein the jaw members 14 and 16 cooperate to grasp tissue therebetween (FIG. 2).

With reference again to FIG. 1, drive assembly 9 including the drive rod 40 are in mechanical communication with the movable handle 26. More particularly, one or more gears, links, springs, or other component(s) that are operably supported and/or disposed within the housing 4 are configured to collectively provide translation of the drive rod 40 along the longitudinal axis “A-A” and though the central passageway 38 defined through the links 32 as a result of proximal movement of the movable handle 26. Drive rod 40 may be made from any suitable material, e.g., metal. In certain embodiments, it may prove advantageous for the drive rod 40 to be relatively flexible. In this instance, the drive rod 40 may be made from a relatively flexible material, e.g., wire, band, cable, etc.

Jaw members 14, 16 are operatively and pivotably coupled to each other and located adjacent the distal end 20 of shaft 18 (FIGS. 1 and 2). For illustrative purposes, the end effector 12 is shown including a bilateral jaw configuration, i.e., both jaw members 14 and 16 are movable. However, the present disclosure contemplates that the end effector 12 may include a unilateral jaw configuration, i.e., jaw member 14 is movable with respect to jaw member 16 that is non-movable or stationary with respect to jaw member 14. Respective electrically conductive seal plates 5 and 7 are operably supported on and secured to jaw housings 28 and 30 of respective the jaw members 14 and 16.

In use, jaw members 14 and 16, initially, are in an open position and the locking tube 19 is in the retracted position (FIG. 1). To position the jaw members 14 and 16 adjacent target tissue, the articulation dials 42a and 42b may be rotated to articulate the shaft 18 transversely across the longitudinal axis “A-A.” Tissue is, subsequently, positioned between the jaw members 14 and 16 and the movable handle 26 may be moved proximally through a clamping stroke. Thereafter, the articulation dials 42a and 42b may be rotated to place the shaft back in-line with the longitudinal axis “A-A.” To maintain the shaft 18 in-line with the longitudinal axis “A-A,” i.e., in an non-articulated configuration, the slide mechanism 15 is moved distally within the elongated slot 13, which, in turn, translates the locking tube 19 distally and over the articulating portion 23 of the shaft 18. The snug fit of the locking tube 19 around the shaft 18 provides a shaft 18 that is as stiff as the locking tube 19, which may be as stiff as a non-articulating shaft. When the locking tube 19 is in the extended position (FIG. 2) the plurality of tendons 34 is in an unloaded state and is not under high tension, as is typically the case with conventional shafts in a locked configuration. As can be appreciated, the tendons 34 retain their ability to stretch and the stiffness of the shaft 18 is not compromised. That is, the stiffness of the shaft 18 is not dependent upon the stiffness of the plurality of tendons 34, but rather the stiffness of the locking tube 19.

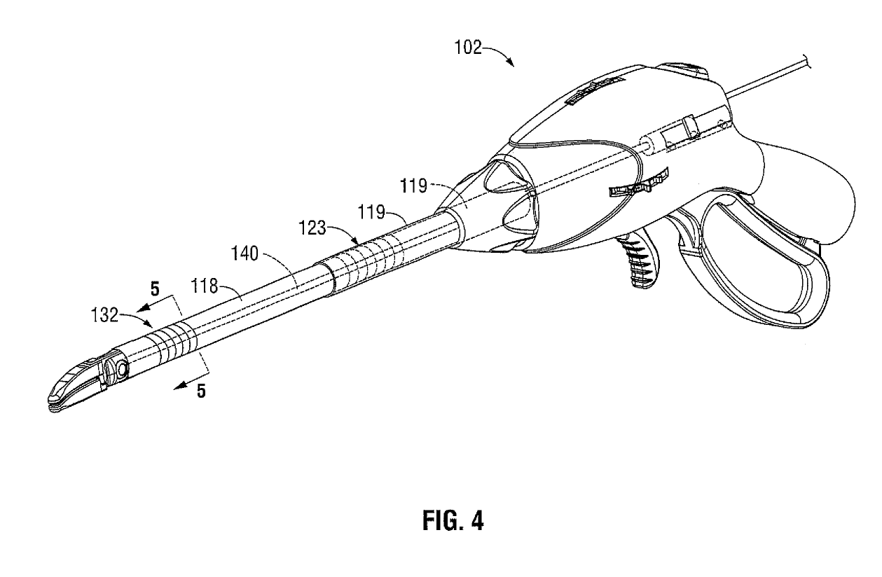

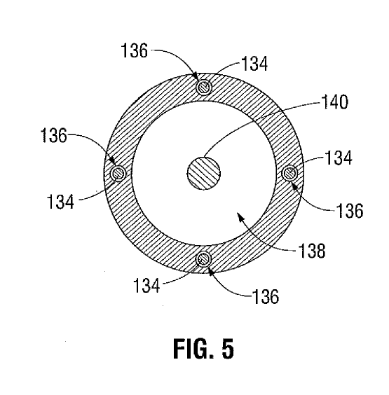

From the foregoing and with reference to the various figure drawings, those skilled in the art will appreciate that certain modifications can also be made to the present disclosure without departing from the scope of the same. For example, in one particular embodiment, it may prove advantageous to have a shaft 118 with an articulating portion 123 that includes a compliant cylindrical extrusion (FIGS. 4 and 5).

The forceps 102 depicted in FIGS. 4 and 5 is substantially similar to the forceps 2. Accordingly, only those features unique to forceps 102 are described in detail.

Unlike articulating portion 23 that includes a plurality of articulating links 32, articulating portion 123 includes a compliant cylindrical extrusion that is operably coupled to the shaft 118. In certain instances, to simplify manufacture of the shaft 118, the entire shaft 118 may be made from the compliant extrusion. In the embodiment illustrated in FIGS. 4 and 5, the shaft 118 is made from the compliant extrusion. An interior of the shaft 118 includes apertures or lumens 136 (FIG. 5) that are formed during the extrusion process of the shaft 118. The lumens 136 may extend along a length of the shaft 118 such that a desired amount of articulation may be achieved. Moreover, an interior of the shaft defines a central lumen 138 configured to receive a drive rod 140 therethrough, see FIG. 5.

An optional second articulating portion 132 is disposed on the shaft 118 adjacent the end effector 112 (FIGS. 4 and 5). Second articulating portion 132 may be a compliant extrusion or may be a plurality of articulating links. The second articulating portion 132 provides an extra degree of articulation when the locking tube 119 is in the extended position over the articulating portion 123 of shaft 118.

A second set of tendons (not explicitly shown) is configured to couple to the second articulation portion 132.

In a fully extended position, the locking tube 119 is covers each of the articulating portions 123 and 132.

Use of the forceps 102 with the locking tube 119 is substantially similar to that of forceps 2 with the locking tube 19 and as such will not be described in greater detail.

In an alternate embodiment, the locking tubes 19, 119 (or an extension thereof) may be configured to translate within the annulus 38, i.e. an internal locking tube configuration. In this instance, the drive rod 40 is positioned within the locking tubes 19, 119, which function as described above, but for translating within the annulus 38 to maintain the shaft 18 in-line with the longitudinal axis “A-A.”

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An endoscopic instrument, comprising:
a housing having shaft extending therefrom that defines a longitudinal axis therethrough, the shaft including an articulating portion disposed thereon;
an end effector assembly operatively connected to a distal end of the shaft configured for treating tissue includes a pair of first and second jaw members; and
a locking tube coaxially disposed on the shaft and axially movable along the longitudinal axis, the locking tube movable along an outer surface of the shaft from a retracted position for articulating the shaft transversely across the longitudinal axis, to an extended position for locking the shaft in a fixed position along the longitudinal axis.

2. An endoscopic instrument according to claim 1, wherein the articulating portion of the shaft is defined by a plurality of articulating links that are operably coupled to a pair of articulation dials of the endoscopic instrument via a plurality of tendons, wherein an outer diameter of the plurality of links is cocylindrical with the shaft.

3. An endoscopic instrument according to claim 1, wherein the plurality of articulating links collectively define a central passageway and a plurality of radially located passageways, the central passageway configured to receive a drive mechanism therethrough and the radially located passageways configured to receive a corresponding tendon of the plurality of tendons.

4. An endoscopic instrument according to claim 2, wherein when the locking tube is in the extended position the plurality of tendons are in an unloaded state.

5. An endoscopic instrument according to claim 2, wherein the locking tube includes an inner diameter configured to contact the outer circumferential surface of the shaft such that the locking tube forms a snug fit around the shaft.

6. An endoscopic instrument according to claim 2, wherein the locking tube is operably coupled to an actuation device operably disposed on the housing of the endoscopic instrument.

7. An endoscopic instrument according to claim 6, wherein the actuation device includes a slide mechanism that is operably coupled to an elongated slot on the housing, the slide mechanism translatable within the elongated slot from a retracted position that corresponds to the locking tube being in the retracted position, to an extended position that corresponds to the locking tube being in the extended position.

8. An endoscopic instrument according to claim 7, wherein the slide mechanism is configured such that the slide mechanism remains in the retracted and extended positions until a predetermined downward force is applied thereto to move the slide mechanism from the retracted and extended positions and vice versa.

9. An endoscopic instrument according to claim 1, wherein the articulating portion of the shaft is defined by a compliant cylindrical extrusion including multiple lumens oriented about a central lumen coaxial with an extrusion axis.

10. An endoscopic instrument according to claim 1, wherein the compliant cylindrical extrusion is made from plastic.

11. An endoscopic instrument according to claim 1, wherein at least one of the first and second jaw members being movable relative to other jaw member from an open position, wherein the first and second jaw members are disposed in spaced relation relative to one another, to a clamping position, wherein the first and second jaw members cooperate to grasp tissue therebetween.

12. An endoscopic instrument, comprising:
a housing having shaft extending therefrom that defines a longitudinal axis therethrough, the shaft including a plurality of articulating links in operable communication with a pair of articulation dials of the endoscopic instrument via a plurality of tendons, wherein an outer diameter of the plurality of articulating links is cocylindrical with shaft;
an end effector assembly operatively connected to a distal end of the shaft configured to treat tissue includes a pair of first and second jaw members; and
a locking tube coaxially supported on the shaft and selectively movable therealong upon actuation of an actuation device disposed on the housing, the locking tube movable along the outer surface of the shaft from a retracted position for articulating the shaft transversely across the longitudinal axis, to an extended position for locking the shaft in a fixed position along the longitudinal axis.

13. An endoscopic instrument according to claim 12, wherein the plurality of articulating links collectively define a central passageway and a plurality of radially located passageways, the central passageway configured to receive a drive mechanism therethrough and the radially located passageways configured to receive a corresponding tendon of the plurality of tendons.

14. An endoscopic instrument according to claim 12, wherein when the locking tube is in the extended position the plurality of tendons are in an unloaded state.

15. An endoscopic instrument according to claim 12, wherein the locking tube includes an inner diameter configured to contact the outer circumferential surface of the shaft such that the locking tube forms a snug fit around the shaft.

16. An endoscopic instrument according to claim 12, wherein the actuation device includes a slide mechanism that is operably coupled to an elongated slot on the housing, the slide mechanism translatable within the elongated slot from a retracted position that corresponds to the locking tube being in the retracted position, to an extended position that corresponds to the locking tube being in the extended position.

17. An endoscopic instrument according to claim 16, wherein the slide mechanism is configured such that the slide mechanism remains in the retracted and extended positions until a predetermined downward force is applied thereto to move the slide mechanism from the retracted and extended positions and vice versa.

18. An endoscopic instrument according to claim 11, wherein the articulating portion of the shaft is defined by a compliant cylindrical extrusion including multiple lumens oriented about a central lumen coaxial with an extrusion axis.

19. An endoscopic instrument according to claim 18, wherein the compliant cylindrical extrusion is made from plastic.

20. An endoscopic instrument according to claim 12, wherein at least one of the first and second jaw members being movable relative to other jaw member from an open position, wherein the first and second jaw members are disposed in spaced relation relative to one another, to a clamping position, wherein the first and second jaw members cooperate to grasp tissue therebetween.
